(19) United States
(12) Patent Application Publication
(10) Pub. No.: US 2009/0311264 A1
(43) Pub. Date: Dec. 17, 2009

(54) Use of Disorazoles and Their Derivatives for the Treatment of Benign and Malignant Oncoses

(75) Inventors: Herbert Irschik, Wolfenbuttel (DE); Rolf Jansen, Braunschweig (DE); Florenz Sasse, Braunschweig (DE); Silke Baasner, Schoneck (DE); Peter Schmidt, Schoneck (DE); Eckhard Gunther, Maintal (DE)

Correspondence Address: GOODWIN PROCTER LLP;ATTN: PATENT ADMINISTRATOR, 620 Eighth Avenue, NEW YORK, NY, 10018, US

(73) Assignee: AEterna Zentaris GmbH, Frankfurt (DE)

(21) Appl. No.: 12/263,981

(22) Filed: Apr. 7, 2009

Related U.S. Application Data
(63) Continuation of application No. 10/646,904, filed on Aug. 22, 2003.

(51) Int. Cl. A61K 31/424; A61P 35/00; A61K 31/437; A61K 31/704; A61K 31/675; A61K 31/519; A61K 39/395; A61K 31/506; A61K 31/5377

(52) U.S. Cl. 424/141.1; 514/375; 514/283; 514/34; 514/110; 514/249; 424/130.1; 514/252.18; 514/234.5

(57) ABSTRACT

The invention relates to disorazoles of the general formula I, which are employed as medicaments, preferably for the treatment of oncoses, in particular in the case of pharmaceutical resistance to other active compounds and in the case of metastasizing carcinoma. The possible uses are not restricted to oncoses.

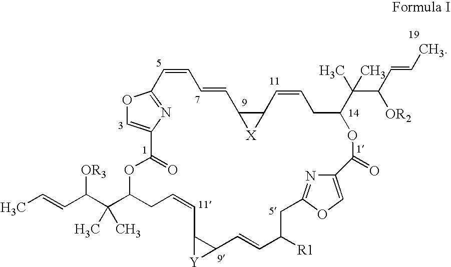

### CROSS-REFERENCE TO RELATED APPLICATIONS

This patent application is a continuation of and claims priority to co-pending U.S. patent application Ser. No. 10/646,904, filed Aug. 22, 2003, the entirety of which is incorporated herein by reference.

### BACKGROUND

For the next few years, a dramatic increase in oncoses and tumor-related cases of death is expected worldwide. In 2001, worldwide approximately 10 million people were suffering from cancer and over 6 million people died from this disease. The development of tumors is a fundamental disease of higher organisms in the plant kingdom, in the animal kingdom and in humans. The generally recognized multistep model of carcinogenesis assumes that as a result of accumulation of a number of mutations in an individual cell this is so modified in its proliferation and differentiation behavior that finally, via benign intermediate stages, a malignant state with metastasis is reached. The term cancer or tumor conceals a clinical picture with more than 200 various individual diseases. Oncoses can proceed in a benign or malignant manner.

The most important tumors are those of the lung, the breast, the stomach, the neck of the uterus, the prostate, the head and neck, the large and small intestine, the liver and the blood system. There are great differences with respect to course, prognosis and therapy behavior. More than the 90% of the cases recognized relate to solid tumors, which in particular in the advanced stage or on metastasis are treatable with difficulty or are untreatable. The three pillars of cancer control are still surgical removal, irradiation and chemotherapy. In spite of great advances it has not yet been possible to develop medicaments which bring about a marked prolongation of the survival time or even a complete cure in the widespread solid tumors. It is therefore meaningful to invent novel medicaments for the control of cancer.

### BRIEF DESCRIPTION OF THE DRAWINGS

FIG. 1 illustrates the results of the in vivo treatment experiment with disorazole E1 in the NCI-H460 tumor xenograft.

### DESCRIPTION OF THE INVENTION

The present invention relates to disorazole—with the exception of disorazole A1—and derivatives of the disorazoles, and to their use as medicaments, in particular for the treatment of benign and malignant tumors in humans and mammals.

It has now surprisingly been found that the disorazoles E1 and D1 in particular possess an outstanding cytotoxic action on various human tumor cell lines. In nano- and picomolar concentrations, the division, inter alia, of ovarian carcinoma, prostate carcinoma, glioblastoma, lung carcinoma and breast cancer cells is inhibited. The action of the disorazoles E1 and D1 is in this case cell cycle-dependent, even in nanomolar concentrations the cell cycle is held in the G2/M phase and the cancer cells are forced into apoptosis. It has further been possible to show that the antiproliferative action of the disorazoles claimed is based, inter alia, on an effective inhibition of tubulin polymerization. Disorazole E1 is in particular also highly active against paclitaxel- and vindesine-resistant cell lines. It was inventively possible to show that disorazole E1 is highly potent with respect to biological action and thus use as an active compound in a medicament for the control of cancers is possible.

This matters in particular, since disorazole Al is unsuitable for use as a cytostatic (G. Hoefle, annual report 1999/2000 of the Gesellschaft für Biotechnologische Forschung [Association for Biotechnological Research] GBF, p. 103).

In a therapeutic experiment, using, for example, NCI-H460 tumor xenograft-bearing nude mice—but not restricted thereto—it was possible to observe, however, for disorazole E1 administered i.v, a significant reduction in tumor growth even at doses which produced no weight decrease or perhaps even mortality.

Natural substances are an important source for novel lead structures in pharmaceutical research and are in some cases also directly suitable for the development of a novel medicament (Y.-Z. Shu, J. Nat. Prod., 1998, 61, 1053-1071). It is known that many natural substances possess strongly cytotoxic action (V. J. Ram, S. Kumari, DNP, 2001, 14(8), 465-482).

It is known that natural substances of the group consisting of the disorazoles are isolated from the bacterium of the strain Sorangium cellulosum So ce12 (R. Jansen, H. Irschik, H. Reichenbach, V. Wray, G. Höfle, Liebigs Ann. Chem., 1994, (8), 759-773). In total, 29 disorazoles have been isolated and characterized physicochemically. For the disorazole A1, it was reported that it possesses an antiproliferative action in cell models (H. Irschik, R. Jansen, K. Gerth, G. Höfle, H. Reichenbach, J. Antibiot. 1995, 48(1), 31-35; Y. A. Elnakady, Dissertation, T. U. Braunschweig, 2001). Use for the treatment of oncoses was, however, neither disclosed nor suggested. A biological investigation of the other disorazoles was not carried out.

The compounds according to the invention are suitable, without being restricted thereto, for employment as medicaments for the treatment of benign and malignant oncoses or other antiproliferative disorders in humans and animals. In principle, the compounds according to the invention are suitable for the control of all disorders which are based on the uncontrolled and rapid division of cells and thereby cause pathological conditions. The compounds according to the invention can be employed as an individual substance or in combination with further cytotoxic substances, e.g. cisplatin, carboplatin, doxorubicin, ifosfamide, cyclophosphamide, 5-FU, methotrexate and in particular in combination with inhibitors of signal transduction, such as, for example, Herceptin, Glivec or Iressa, but not restricted thereto.

Synthetic and semisynthetic analogs of the disorazoles also possess antiproliferative action. By means of specific modification of the molecular shape, important properties such as biological inhibitory action, stability and biophysical properties can be modulated. In this manner, therapeutically valuable derivatives of the starting compounds are obtainable. A further aim of the derivatization consists in moderating possible toxic side effects.

The compounds according to the invention can be administered as liquid pharmaceutical forms. This is carried out in the manner suitable in each case in the form of solutions or suspensions.

The compounds according to the invention can be administered in a suitable administration form, preferably into an artery, intraarterally as an injection; into a vein, intravenously as an injection or infusion; into the skin, intracutaneously as an injection; under the skin, subcutaneously as an injection; into the muscle, intramuscularly as an injection; into the abdominal cavity, intraperitoneally as an injection or infusion.

If the compounds of the general formula I according to the invention have at least one asymmetric center, they can be present in the form of their racemates, in the form of the pure enantiomers and/or diastereomers or in the form of mixtures of these enantiomers and/or diastereomers, namely both in substance and as pharmaceutically acceptable salts of these compounds. The mixtures can be present in any desired mixing ratio of the stereoisomers. If possible, the configurations of each of the double bonds in the compounds according to the invention can independently of one another in each case be E or Z.

If possible, the compounds according to the invention can be present in the form of the tautomers.

According to one embodiment, the invention relates to compounds of the general formula I:

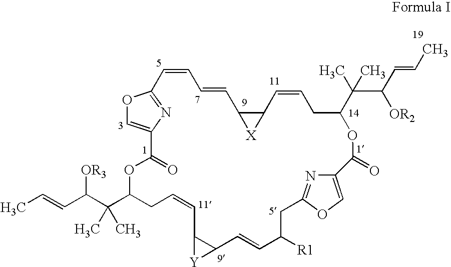

in which independently of one another

R1 is:
(i) hydrogen
(ii) OR4
(iii) part of a double bond to C5′

R2, R3 and R4 are:
(i) hydrogen
(ii) unsubstituted or substituted (C₁-C₆)-alkyl,
(iii) (C₁-C₄)-alkyl substituted by one or more fluorine atoms, preferably a trifluoromethyl group,
(iv) unsubstituted or substituted (C₁-C₄)-alkyl-(C₆-C₁₄)-aryl, unsubstituted or substituted (C₁-C₄)-alkyl-heteroaryl,
(v) (C₁-C₄)-alkoxycarbonyl, (C₁-C₄)-alkylaminocarbonyl (C₁-C₄)-alkylaminothiocarbonyl, (C₁-C₆)-alkyl-carbonyl or (C₁-C₆)-alkoxycarbonyl-(C₁-C₆)-alkyl,
it being possible for the substitution of the alkyl radical by F, Cl, Br, I, CN, NH₂, NH-(C₁-C₂₀)-alkyl, NH-(C₃-C₁₂)-cycloalkyl, OH, O-(C₁-C₂₀)-alkyl to take place singly or, on identical or different atoms, multiply by identical or different substituents, and it being possible for the substitution of an aryl radical by F, Cl, Br, I, CN, NH₂, NH-(C₁-C₂₀)-alkyl, OH, O-(C₁-C₂₀)-alkyl and/or (C₃-C₈)-heterocyclyl having 1 to 5 heteroatoms, preferably nitrogen, oxygen, sulfur to take place singly or, on identical or different atoms, multiply by identical or different substituents,
   and

X, Y are: in each case individually independently of one another or together oxygen, sulfur, two vicinal hydroxyl groups, two vicinal methoxy groups, part of a double bond, a compound being excluded in which R1 is methoxy, R2, R3 are hydrogen, X is oxygen and Y is the part of a double bond.

The term “aryl” means for the purpose of this invention aromatic hydrocarbons, inter alia phenyls, naphthyls and anthracenyls. The radicals may also be fused to other saturated, (partially) unsaturated or aromatic ring systems.

The term “heteroaryl” stands for a 5-, 6- or 7-membered cyclic aromatic radical which comprises at least 1, where appropriate also 2, 3, 4 or 5, heteroatoms, the heteroatoms being identical or different.

The heterocycle may also be part of a bi- or polycyclic system. Preferred heteroatoms are nitrogen, oxygen and sulphur. It is preferred for the heteroaryl radical to be selected from the group comprising pyrrolyl, furyl, thienyl, thiazolyl, oxazolyl, isoxazolyl, pyrazolyl, imidazolyl, pyridinyl, pyrimidinyl, pyrazinyl, indolyl, indolizinyl, quinolinyl, isoquinolinyl, quinazolinyl, quinoxalinyl, carbazolyl, phenazinyl, phenothiazinyl, acridinyl.

The most preferred compounds according to the general formula I are those which are encountered in the following selection:

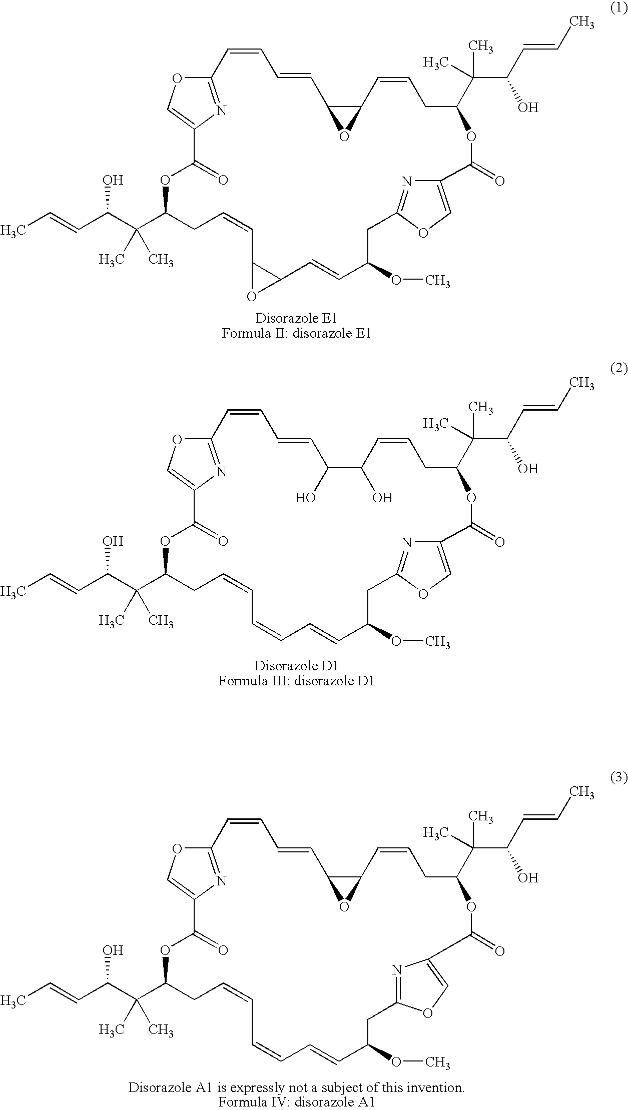

The invention will be illustrated in greater detail with the aid of the following examples, without being restricted thereto.

### EXAMPLES

### Use Possibilities

### Example 1

Disorazoles such as, for example, disorazole E1 are preferred as an active compound in a ready-to-use medicament for the treatment of malignant oncoses such as breast cancer, lung cancer, ovarian cancer, skin cancer, prostate cancer, colonic cancer, renal cell cancer, hepatic cancer, pancreatic cancer and cancers of the brain.

In a preferred administration form, the active compound is present as a lyophilizate together with the excipients known to the person skilled in the art in an injection bottle and is dissolved using physiological saline solution before use, then diluted in an injection bag and administered to the patient with the aid of a cannula into the vein. The dose, depending on the stage of the oncosis and the state of health of the patient, is between 0.1 mg and 100 mg of active compound per m². The infusion period depends on the objective criteria of the disease.

### Example 2

Use of disorazoles such as, for example, disorazole E1 as an active compound in a ready-to-use medicament for the treatment of inflammatory diseases. These include, for example, inflammatory airway diseases such as bronchial asthma, allergic rhinitis, allergic conjunctivitis, atopic dermatitis, eczema, allergic angiitis, inflammations mediated by eosinophils such as eosinophilic pneumonia and PIE syndrome (pulmonary infiltration with eosinophilia), urticaria, ulcerative colitis, Crohn's disease and proliferative skin diseases such as psoriasis and keratosis.

### Example 3

Use of disorazoles such as, for example, disorazole E1 as an active compound in a ready-to-use medicament having immunomodulatory action for the treatment of immune and autoimmune diseases. Such diseases can include, for example, joint inflammations such as arthritis and rheumatoid arthritis and other arthritic diseases such as rheumatoid spondylitis and osteoarthritis. Further possibilities of use are the treatment of patients who are suffering from sepsis, septic shock, Gram-negative sepsis, toxic shock syndrome, respiratory distress syndrome, asthma and other chronic pulmonary diseases, bone resorption diseases or transplant rejection reactions or other autoimmune diseases, such as lupus erythematosus, multiple sclerosis, glomerulonephritis and uveitis, insulin-dependent diabetes mellitus and chronic demyelinization.

### Example 4

Use of disorazoles such as, example disorazole E1 as an active compound in a ready-to-use medicament which can be employed for the therapy of infections such as virus infections and parasite infections, for example for the therapy of malaria, infection-related fever, infection-related muscle pain, HIV infections (AIDS) and cachexias.

### Production

For the administration of the compounds according to the invention, parenteral, transdermal, topical, inhalative and intranasal preparations are preferably suitable.

The production, filling and sealing of the preparations is carried out under the customary antimicrobial and aseptic conditions.

In addition to at least one constituent according to the invention, the pharmaceutical forms, depending on the pharmaceutical form employed, optionally contain excipients, such as, inter alia, solvents, solution accelerators, solubilizers, emulsifiers, wetting agents, antifoams, gel-forming agents, thickeners, buffers, salt-forming agents, preservatives, antioxidants, colorants, taste and odor corrigents. The choice of the excipients and the amounts thereof to be employed depends on the pharmaceutical form chosen and is adapted to the formulations known to the person skilled in the art.

The medicaments according to the invention can be administered in a suitable administration form to the skin, epicutaneously as a solution, suspension, emulsion, foam, ointment, paste or patch; via the nasal mucosa, nasally as drops, ointment, or spray; via the bronchial and alveolar epithelium, pulmonarily or by inhalation as an aerosol or inhalant; via the conjunctiva, conjunctivally as eye drops, eye ointment, eye tablets, lamellae or eye lotion; into an artery, intraarterially as an injection; into a vein, intravenously as an injection or infusion, paravenously as an injection or infusion; into the skin, intracutaneously as an injection or implant; under the skin, subcutaneously as an injection or implant; into the muscle, intramuscularly as an injection or implant; into the abdominal cavity, intraperitoneally as an injection or infusion.

In tumor therapy, the compounds of the general formula I according to the invention can be employed as an individual substance or in combination with further cytotoxic substances, such as, for example, paclitaxel, docetaxel, vincristine, vindesine, cisplatin, carboplatin, doxorubicin, ifosfamide, cyclophosphamide, 5-FU, methotrexate or in combination with immunomodulators or antibodies and in particular in combination with inhibitors of signal transduction, such as, for example, Herceptin, Glivec or Iressa.

### Example 5

Preparations for the parenteral administration of disorazoles such as, for example, disorazole E1, can be present in separate dose unit forms such as, for example, ampoules or vials. Preferably, solutions of the active compound are used, preferably aqueous solutions and especially isotonic solutions or alternatively suspensions. These injection forms can be made available as a ready-to-use preparation or are prepared only directly before use by mixing the active compound, for example the lyophilizate, if appropriate with further solid carriers, with the desired solvent or suspending agent.

### Example 6

Preparations for the intranasal administration of disorazoles such as, for example, disorazole E1, can be present as aqueous or oily solutions or as aqueous or oily suspensions. They can also be present as lyophilizates, which are prepared before use using the suitable solvent or suspending agent.

### Biological Actions of the Compounds According to the Invention

### Example 7

### Antiproliferative Action on Various Tumor Cell Lines

The compounds according to the invention were investigated for their antiproliferative activity in a proliferation test on established tumor cell lines (D. A. Scuderio et al. Cancer Res. 1988, 48, 4827-4833). The test used determines the cellular dehydrogenase activity and makes possible a determination of the cell vitality and indirectly of the cell count. The cell lines used are the human cervical carcinoma cell line KB/HeLa (ATCC CCL17), the ovarian adenocarcinoma cell line SKOV-3 (ATCC HTB77), the human glioblastoma cell line SF-268 (NCI 503138), the lung carcinoma cell line NCI-H460 (NCI 503473) and the human colon adenocarcinoma cell line RKOP 27.

The cytotoxic or growth-inhibiting activity of the compounds described is shown in table 1. The results show a very potent inhibition of the proliferation of selected tumor cell lines by the substances mentioned.

TABLE 1
Inhibition of proliferation by substances according to the
invention in the XTT cytotoxicity test on human tumor cell lines

| | XTT proliferation assay, EC50 in [μg/ml] | | | | |
|---|---|---|---|---|---|
| Example | KB/Hela | SKOV3 | SF-268 | NCl-H460 | RKOP 27 |
| Disorazole E1 | 0.00007 | 0.00002 | 0.00017 | 0.00004 | 0.00006 |
| Disorazole D1 | <0.0001 | <0.0001 | 0.00035 | <0.0001 | 0.0003 |
| Disorazole A1 | 0.00015 | 0.0002 | 0.00027 | 0.00015 | 0.00025 |
| Paclitaxel | 0.01 | 0.01 | 0.01 | 0.01 | |
| Vindesine | 0.002 | 0.002 | 0.005 | 0.006 | |

### Example 8

### Antiproliferative Action on MDR Tumor Cell Lines

For further characterization, the substances according to the invention were investigated against multi-drug-resistant cell lines (MDR) in comparison to the nonresistant wild-type cell lines. The cell lines investigated are the acute myeloid leukemia cell line LT1 and the resistant line LT12/mdr. Moreover, the murine P388 cell line (methylcholanthrene-induced lymphoid neoplasm) and the doxorubicin-resistant P388 were used as test systems.

The results are shown in summarized form in table 2 below:

TABLE 2
Inhibitory action of disorazole E1 and reference substances in the
XTT proliferation test on nonresistant and resistant tumor cell
lines.

| | XTT proliferation assay, EC₅₀ in [μg/ml] | | | |
|---|---|---|---|---|
| Substance | LT12 | LT12MDR | P388 | P388ADR |
| Disorazole E1 | 0.0001 | 0.004 | 0.0004 | 0.001 |
| Paclitaxel | 0.005 | 0.340 | 0.035 | >3.16 |
| Vindesine | 0.0009 | 0.222 | 0.009 | 0.94 |

Disorazole E1 shows a very potent inhibitory action on all cell lines tested, while in the case of the classical tubulin inhibitors such as paclitaxel or vincristine a greatly decreased action and cross resistances to the MDR1 cell lines can be detected.

### Example 9

### Inhibition of the Polymerization of Tubulin

The substances were tested in an in-vitro test for inhibition of the polymerization of bovine β-tubulin (D. M. Bollag et al. Cancer Res. 1995, 55, 2325-2333). In this test, tubulin purified by cycles of polymerization and depolymerization is employed, and is polymerized by addition of GTP and warming. The EC₅₀ values of the inhibition of polymerization of β-tubulin with and without 30% associated proteins (MAPs) are indicated in table 3.

TABLE 3
Inhibition of the polymerization of β-tubulin with 30% MAPs.

| | EC₅₀ in [μg/ml] |
|---|---|
| Substances | with 30% MAPs |
| Disorazole E1 | 1.50 |
| Disorazole D1 | 2.50 |
| Disorazole A1 | 4.80 |
| Vindesine | 0.40 |

experiments: n = 2

The results show that the disorazoles E1 and D1 inhibit tubulin polymerization at low concentrations.

### Example 10

### Cell Cycle Analysis

The cell cycle comprises the development of the cell from one cell generation to the next. During the resting phase (G0) and presynthetic phase (G1), the cell has a diploid chromosome set (2c). In the synthesis phase (S), the amount of DNA is increased by replication. The S phase ends by reaching the premitotic phase (G2M), in which the cell has a reduplicated chromosome complement (4c) and doubled DNA content. In the subsequent, transient mitosis phase (M) the uniform division of the reduplicated chromosomes to two daughter cells occurs, which then in each case again show a diploid DNA content and are in the G01 phase, so that the cell cycle can begin anew.

For the cell cycle analysis, KB/HeLa cells were treated with the test substances in different concentrations (0.1-1000 nM) for 24 hours at 37° C.

The percentage proportion of the cells arrested in the G2/M phase of the cell cycle after treatment with reference substances or selected test substances is shown in table 4 below. The results were evaluated using special analysis software (ModFit™).

TABLE 4
concentration at which 50% of the cells are arrested in the G2/M
phase.

| | EC₅₀ in [nM] (50% cells in |
|---|---|
| Example | G2/M) |
| Disorazole E1 | 1.6 |
| Paclitaxel | 46 |
| Vindesine | 3.0 |

The compounds according to the invention have the highest activities in comparison with the reference compounds. In particular, disorazole E1 inhibits the cell cycle in the G2/M phase in extremely low concentrations.

### Example 11

### In Vivo Results

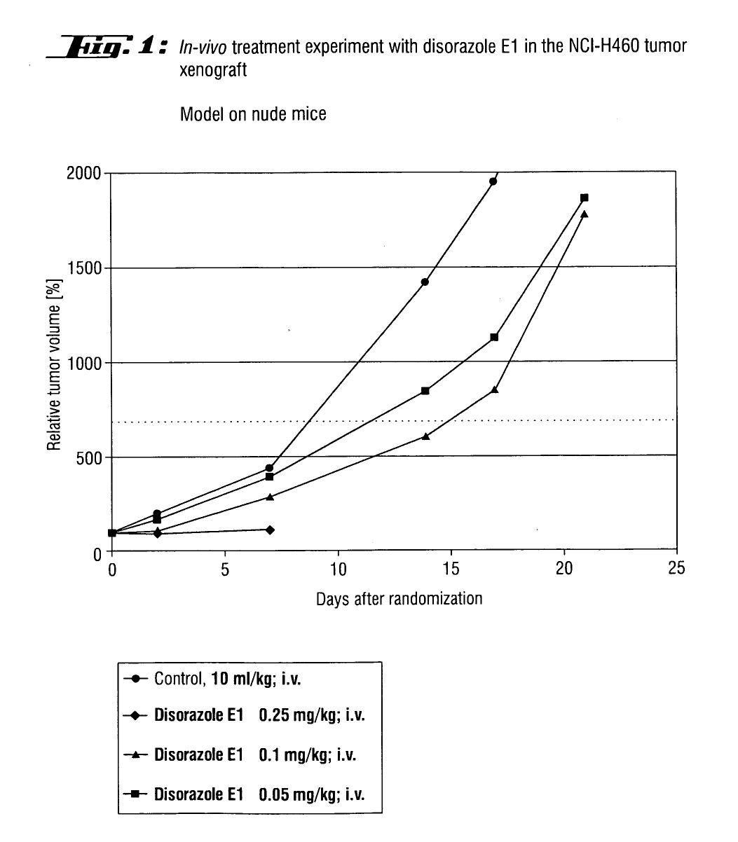

The in-vivo activity of the compounds according to the invention was tested on human and murine xenograft models. In the therapy experiment, with NCI-H460 tumor xenograft-bearing nude mice, it was possible for disorazole E1 administered i.v. to produce a significant reduction of the tumor growth even at doses which produced no significant weight decrease or perhaps even mortality. The results are shown in FIG. 1.

| | |
|---|---|
| Disorazole E1 (D-42805): | 0.25 mg/kg; i.v.: day 0, 7; 8 dead (day |
| | 11, 12, 13) |
| Disorazole E1 (D-42805): | 0.1 mg/kg; i.v.: day 0, 7, 14; no cases of |
| | death |
| Disorazole E1 (D-42805): | 0.05 mg/kg; i.v.: day 0, 7, 14; no cases of |
| | death |

Control: 0.9% strength saline solution containing 3.3% DMSO, 10 ml/kg; n = 8 animals/group

### Example 12

### AMES Test

For the estimation of possible side effects, disorazole E1 was investigated for mutagenicity in a fluctuation assay against the mutant strains TA98 and TA100 of the bacterium Salmonella typhimurium at three concentrations (2.5; 5 and 10 μM). The mutagenicity investigations were further carried out in the presence of the rat liver enzyme S9.

The results are compiled in table 5 below:

TABLE 5
Investigation of disorazole E1 for mutagenicity

| | | | | AMES | AMES |
|---|---|---|---|---|---|
| | Conc. | AMES TA98 | AMES TA98 | TA100 | TA100 |
| Compound | [μM] | without S9 | with S9 | without S9 | with S9 |
| Disorazole E1 | 10 | inactive | inactive | inactive | inactive |
| Disorazole E1 | 5 | inactive | inactive | inactive | inactive |
| Disorazole E1 | 2.5 | inactive | inactive | inactive | inactive |

Disorazole E1 shows no effects under the assay conditions described in the abovementioned concentrations, it is thus AMES test-inactive.

### Example 13

### Influence on Protein Biosynthesis and Nonproliferating Cells

For the estimation of the possible side-effect potential, the influence of disorazole E1 on nonproliferating cells and on the protein biosynthesis was investigated (table 6).

TABLE 6
Influence of disorazole E1 on nonproliferating
cells and on the protein biosynthesis

| | | Surviving cells, | |
|---|---|---|---|
| | | primary human | Protein |
| | | hepatocytes¹ | synthesis² |
| | Conc. | Average, % of | Average, % of |
| Substance | [μM] | control | control |
| Disorazole E1 | 1 | 119.6 | 95.9 |

¹Tested with alamarBlue, human primary hepatocytes, n = 3;
²Tested via the incorporation of 14C-methionine, human hepatocellular carcinoma cells (HepG2), n = 2;

The results of table 6 show that disorazole E1 neither acts negatively on the protein biosynthesis nor on the survival of nonproliferating cells.

1. A method for the treatment of oncoses selected from the group consisting of tumors of the lung, the breast, the stomach, the neck, the uterus, the prostate, the head and neck, the large and small intestine, and the liver and the blood system; ovarian carcinoma, prostate carcinoma; glioblastoma; lung carcinoma; breast cancer; skin cancer; colonic cancer; renal cell cancer; hepatic cancer; pancreatic cancer; cervical cancer; and cancers of the brain, comprising administering a compound of the general formula Ia 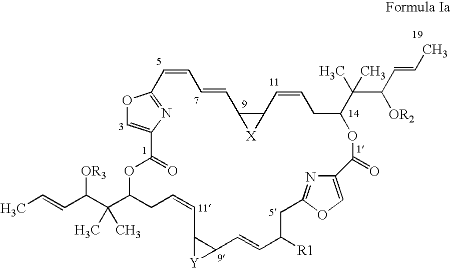
in which independently of one another
R1 is:
(i) hydrogen,
(ii) OR4,
(iii) part of a double bond to C5′,
R2, R3 and R4 are:
(i) hydrogen,
(ii) unsubstituted or substituted (C₁-C₆)-alkyl,
(iii) (C₁-C₄)-alkyl substituted by one or more fluorine atoms, preferably a trifluoromethyl group,
(iv) unsubstituted or substituted (C₁-C₄)-alkyl-(C₆-C₁₄)-aryl, unsubstituted or substituted (C₁-C₄)-alkyl-heteroaryl,
(v) (C₁-C₄)-alkoxycarbonyl, (C₁-C₄)-alkylaminocarbonyl (C₁-C₄)-alkylaminothiocarbonyl, (C₁-C₆)-alkyl-carbonyl or (C₁-C₆)-alkoxycarbonyl-(C₁-C₆)-alkyl,
it being possible for the substitution of the alkyl radical by F, Cl, Br, I, CN, NH₂, NH-(C₁-C₂₀)-alkyl, NH-(C₃-C₁₂)-cycloalkyl, OH, O-(C₁-C₂₀)-alkyl to take place singly or, on identical or different atoms, multiply by identical or different substituents, and it being possible for the substitution of an aryl radical by F, Cl, Br, I, CN, NH₂, NH-(C₁-C₂₀)-alkyl, OH, O-(C₁-C₂₀)-alkyl and/or (C₃-C₈)-heterocyclyl having 1 to 5 heteroatoms, preferably nitrogen, oxygen, sulfur to take place singly or, on identical or different atoms, multiply by identical or different substituents,
and
X, Y are: in each case individually independently of one another or together oxygen, sulfur, two vicinal hydroxyl groups, two vicinal methoxy groups, part of a double bond,
with the proviso that the compound in which R1 is methoxy, R2, R3 are hydrogen, X is oxygen and Y is the part of a double bond is excluded, its tautomers, E/Z isomers, stereoisomers, including the diastereomers and enantiomers, and the physiologically tolerable salts thereof,
alone or in combination with a cytotoxic substance and/or an inhibitor of signal transduction, to an individual in need thereof.

2. A method of inhibiting mitosis in rapidly and uncontrolledly proliferating endogenous cells in humans or animals comprising administering a compound of the general formula Ia 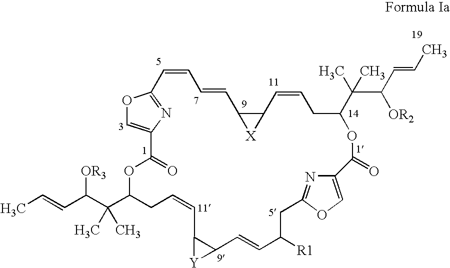
in which independently of one another
R1 is:
(iv) hydrogen,
(v) OR4,
(vi) part of a double bond to C5′,
R2, R3 and R4 are:
(vi) hydrogen,
(vii) unsubstituted or substituted (C₁-C₆)-alkyl,
(viii) (C₁-C₄)-alkyl substituted by one or more fluorine atoms, preferably a trifluoromethyl group,
(ix) unsubstituted or substituted (C₁-C₄)-alkyl-(C₆-C₁₄)-aryl, unsubstituted or substituted (C₁-C₄)-alkyl-heteroaryl,
(x) (C₁-C₄)-alkoxycarbonyl, (C₁-C₄)-alkylaminocarbonyl (C₁-C₄)-alkylaminothiocarbonyl, (C₁-C₆)-alkyl-carbonyl or (C₁-C₆)-alkoxycarbonyl-(C₁-C₆)-alkyl,
it being possible for the substitution of the alkyl radical by F, Cl, Br, I, CN, NH₂, NH-(C₁-C₂₀)-alkyl, NH-(C₃-C₁₂)-cycloalkyl, OH, O-(C₁-C₂₀)-alkyl to take place singly or, on identical or different atoms, multiply by identical or different substituents, and it being possible for the substitution of an aryl radical by F, Cl, Br, I, CN, NH₂, NH-(C₁-C₂₀)-alkyl, OH, O-(C₁-C₂₀)-alkyl and/or (C₃-C₈)-heterocyclyl having 1 to 5 heteroatoms, preferably nitrogen, oxygen, sulfur to take place singly or, on identical or different atoms, multiply by identical or different substituents,
and
X, Y are: in each case individually independently of one another or together oxygen, sulfur, two vicinal hydroxyl groups, two vicinal methoxy groups, part of a double bond,
with the proviso that the compound in which R1 is methoxy, R2, R3 are hydrogen, X is oxygen and Y is the part of a double bond is excluded,
its tautomers, E/Z isomers, stereoisomers, including the diastereomers and enantiomers, and the physiologically tolerable salts thereof, to a human or animal in need thereof.

3. A method for the treatment of benign or malignant oncoses in humans or animals selected from the group consisting of breast cancer, lung cancer, ovarian cancer, skin cancer, prostate cancer, colonic cancer, renal cell cancer, hepatic cancer, pancreatic cancer and cancers of the brain; inflammatory diseases selected from the group consisting of bronchial asthma, allergic rhinitis, allergic conjunctivitis, atopic dermatitis, eczema, and allergic angiitis; inflammations mediated by eosinophils such as eosinophilic pneumonia; pulmonary infiltration with eosinophilia syndrome (PIE syndrome); urticaria; ulcerative colitis; Crohn's disease; psoriasis; or keratosis, comprising administering a compound of the general formula Ia 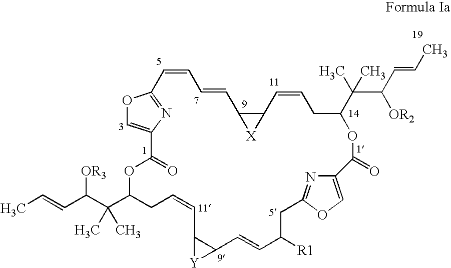
in which independently of one another
R1 is:
(vii) hydrogen,
(viii) OR4,
(ix) part of a double bond to C5′,
R2, R3 and R4 are:
(xi) hydrogen,
(xii) unsubstituted or substituted (C₁-C₆)-alkyl,
(xiii) (C₁-C₄)-alkyl substituted by one or more fluorine atoms, preferably a trifluoromethyl group,
(xiv) unsubstituted or substituted (C₁-C₄)-alkyl-(C₆-C₁₄)-aryl, unsubstituted or substituted (C₁-C₄)-alkyl-heteroaryl,
(xv) (C₁-C₄)-alkoxycarbonyl, (C₁-C₄)-alkylaminocarbonyl (C₁-C₄)-alkylaminothiocarbonyl, (C₁-C₆)-alkyl-carbonyl or (C₁-C₆)-alkoxycarbonyl-(C₁-C₆)-alkyl,
it being possible for the substitution of the alkyl radical by F, Cl, Br, I, CN, NH₂, NH-(C₁-C₂₀)-alkyl, NH-(C₃-C₁₂)-cycloalkyl, OH, O-(C₁-C₂₀)-alkyl to take place singly or, on identical or different atoms, multiply by identical or different substituents, and it being possible for the substitution of an aryl radical by F, Cl, Br, I, CN, NH₂, NH-(C₁-C₂₀)-alkyl, OH, O-(C₁-C₂₀)-alkyl and/or (C₃-C₈)-heterocyclyl having 1 to 5 heteroatoms, preferably nitrogen, oxygen, sulfur to take place singly or, on identical or different atoms, multiply by identical or different substituents,
and
X, Y are: in each case individually independently of one another or together oxygen, sulfur, two vicinal hydroxyl groups, two vicinal methoxy groups, part of a double bond,
with the proviso that the compound in which R1 is methoxy, R2, R3 are hydrogen, X is oxygen and Y is the part of a double bond is excluded, its tautomers, E/Z isomers, stereoisomers, including the diastereomers and enantiomers, and the physiologically tolerable salts thereof to a human or animal in need of such treatment.

4. The method as claimed in claim 3 wherein the oncos is breast cancer, ovarian cancer, lung cancer, skin cancer, prostate cancer, renal cell cancer, hepatic cancer, pancreatic cancer, colonic cancer or brain cancer in humans.

5. The method of claim 3, wherein the compound of formula Ia is administered in combination with another antitumor agent.

6. The method of claim 3, wherein the compound of formula Ia is administered in combination with paclitaxel, docetaxel, vincristine, vindesine, cisplatin, carboplatin, doxorubicin, ifosfamide, cyclophosphamide, 5-FU, methotrexate or in combination with an immunomodulator or antibody or in combination with a signal transduction inhibitor.

7. The method of claim 6, wherein the signal transduction inhibitor is Herceptin, Glivec or Iressa.

8. A method for the treatment of a tumor disease selected from the group consisting of prostate carcinoma, lung carcinoma, leukemia, paclitaxel- and vindesine-resistant tumors, and doxorubicin-resistant tumors, tumors of the stomach, tumors of the neck, tumors of the uterus, tumors of the head and neck, tumors of the large and small intestine, skin cancer, breast cancer, ovarian cancer, cervical cancer, pancreatic cancer, prostate cancer, hepatic cancer, renal cancer, skin cancer, cancers of the brain, cervical carcinoma, ovarian adenocarcinoma, glioblastoma, lung carcinoma, breast cancer, colon cancer and blood cancer, comprising administering a disorazole compound of the general formula Ia: 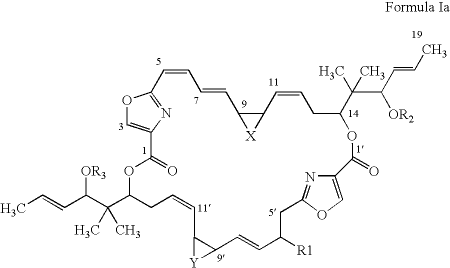
in which independently of one another
R1 is:
(x) hydrogen,
(xi) OR4,
(xii) part of a double bond to C5′,
R2, R3 and R4 are:
(xvi) hydrogen,
(xvii) unsubstituted or substituted (C₁-C₆)-alkyl,
(xviii) (C₁-C₄)-alkyl substituted by one or more fluorine atoms, preferably a trifluoromethyl group,
(xix) unsubstituted or substituted (C₁-C₄)-alkyl-(C₆-C₁₄)-aryl, unsubstituted or substituted (C₁-C₄)-alkyl-heteroaryl,
(xx) (C₁-C₄)-alkoxycarbonyl, (C₁ -C₄)-alkylaminocarbonyl (C₁-C₄)-alkylaminothiocarbonyl, (C₁-C₆)-alkyl-carbonyl or (C₁-C₆)-alkoxycarbonyl-(C₁-C₆)-alkyl,
it being possible for the substitution of the alkyl radical by F, Cl, Br, I, CN, NH₂, NH-(C₁-C₂₀)-alkyl, NH-(C₃-C₁₂)-cycloalkyl, OH, O-(C₁-C₂₀)-alkyl to take place singly or, on identical or different atoms, multiply by identical or different substituents, and it being possible for the substitution of an aryl radical by F, Cl, Br, I, CN, NH₂, NH-(C₁-C₂₀)-alkyl, OH, O-(C₁-C₂₀)-alkyl and/or (C₃-C₈)-heterocyclyl having 1 to 5 heteroatoms, preferably nitrogen, oxygen, sulfur to take place singly or, on identical or different atoms, multiply by identical or different substituents,
and
X, Y are: in each case individually independently of one another or together oxygen, sulfur, two vicinal hydroxyl groups, two vicinal methoxy groups, part of a double bond,
with the proviso that the compound in which R1 is methoxy, R2, R3 are hydrogen, X is oxygen and Y is the part of a double bond is excluded, its tautomers, E/Z isomers, stereoisomers, including the diastereomers and enantiomers, and the physiologically tolerable salts thereof, to an individual in need of such treatment alone or in combination with a cytotoxic substance and/or an inhibitor of signal transduction.
